(19) United States
(12) Patent Application Publication
(10) Pub. No.: US 2023/0271898 A1
(43) Pub. Date: Aug. 31, 2023

(54) ETHYLENE OLIGOMERISATION PROCESS COMPRISING IN SITU PREPARATION OF THE CATALYTIC COMPOSITION

(71) Applicant: IFP Energies Nouvelles, Rueil-Malmaison (FR)

(72) Inventors: Typhene MICHEL, Rueil-Malmaison Cedex (FR); Pierre-Alain BREUIL, Rueil-Malmaison Cedex (FR); Lionel MAGNA, Rueil-Malmaison Cedex (FR)

(73) Assignee: IFP Energies Nouvelles, Rueil-Malmaison (FR)

(21) Appl. No.: 18/018,086

(22) PCT Filed: Jul. 13, 2021
(86) PCT No.: PCT/EP2021/069498

§ 371 Date: Jan. 26, 2023

(30) Foreign Application Priority Data
Jul. 30, 2020 (FR) 2008093

(51) Int. Cl. C07C 2/26; B01J 31/22; B01J 31/02

(52) U.S. Cl. CPC C07C 2/26; B01J 31/22; B01J 31/0244; C07C 2531/14; C07C 2531/02

(57) ABSTRACT

The invention relates to a process for oligomerization of ethylene, preferably for selective trimerization of ethylene to hex-1-ene, comprising simultaneously bringing ethylene into contact with the components of a catalytic composition based on chromium.

### TECHNICAL FIELD OF THE INVENTION

The invention relates to a process for oligomerization of ethylene, preferably for selective trimerization of ethylene to hex-1-ene, comprising simultaneously bringing ethylene into contact with the components of a catalytic composition based on chromium.

### PRIOR ART

The trimerization of ethylene to hex-1-ene by a homogeneous catalyst based on chromium has been studied since the 1980s. Among the catalytic systems known to result in the selective production of hex-1-ene, mention may for example be made of the systems described in patents U.S. Pat. Nos. 5,198,563, 5,288,823, 5,382,738, EP 608 447, EP 611 743, EP 614 865. These catalysts are prepared from a chromium salt and a metal amide, a pyrrolide in particular, activated in situ by an alkylaluminum or a mixture of alkylaluminum and chloroalkylaluminum.

It is commonly observed during the operation of these chromium-based catalytic systems that the activity and the productivity of the catalytic system may decrease. Moreover, harsh reaction conditions, such as high temperatures, may increase the productivity of the catalyst, but this generally leads to a shorter service life of the catalytic system and sometimes even to a loss of the initial selectivity of the catalyst. The use of additives may then be envisaged to counterbalance this phenomenon and ensure the good stability of the catalyst and the maintenance of its performance, in particular at high temperature.

Document US 2001/0053742 describes the effect of the order of addition of the various constituents of a catalytic composition comprising chromium, a pyrrole derivative and an alkylaluminum on the formation of polyethylene during the ethylene trimerization reaction. This document specifies that the contacting of the various constituents during the preparation of the catalytic composition must be carried out under an inert atmosphere and at a temperature of between 10° C. and 40° C. so as to minimize the formation of particles and to improve the performance of the catalytic composition thus prepared.

Surprisingly, the applicant has discovered a new ethylene oligomerization process that dispenses with the drawbacks linked to the prior preparation of the catalytic composition and makes it possible to obtain excellent performance in terms of selectivity and activity. In particular, said process comprises bringing the various components of the catalytic composition into contact in situ, i.e. in the reactor in the presence of ethylene at a temperature of between 100° C. and 190° C.

### SUBJECT OF THE INVENTION

The present invention relates to an ethylene oligomerization process comprising, at a temperature between 90° C. and 190° C., simultaneously bringing ethylene into contact with the following components
a metal precursor of chromium
a pyrrole derivative
an aluminum-based compound of general formula AlR²R³R⁴ wherein the R², R³ and R⁴ groups, which may be identical or different, are chosen independently from a hydrogen and C₁-C₂₀ alkyl, C₁-C₂₀ alkoxy and C₅-C₃₀ aryloxy groups,
at least one halogenated aluminum compound corresponding to the general formula AlₙR⁵ₒYₚ, wherein R⁵ is a C₁-C₂₀ alkyl group, Y is a halogen, n is an integer from 1 to 2, o is an integer from 1 to 3 and p is an integer from 1 to 3;
an aromatic additive;
optionally a solvent,
   said components forming in situ a catalytic composition.

The process according to the present invention advantageously makes it possible to form in situ the catalytic composition at high temperature and in the presence of ethylene.

One advantage of the process according to the present invention is that of significantly improving the stability of the catalytic formulation at high temperature while maintaining a high activity and a high selectivity in favor of hex-1-ene.

Preferably, the aluminum-based compound and the halogenated aluminum compound are mixed prior to them being brought into contact with ethylene.

Preferably, the aluminum-based compound and the halogenated aluminum compound are mixed before being mixed with the pyrrole derivative prior to them being brought into contact with ethylene.

Preferably, the aluminum-based compound and the halogenated aluminum compound, the pyrrole derivative and the aromatic additive are mixed prior to them being brought into contact with ethylene.

Preferably, the metal precursor of chromium and the aromatic additive are mixed prior to them being brought into contact with ethylene.

Preferably, not all the components are mixed before being brought into contact with ethylene. In this embodiment, all the components are mixed at the time they are brought into contact with ethylene.

Preferably, the temperature for bringing the components into contact with ethylene is between 95° C. and 185° C., preferably between 100° C. and 160° C.

Preferably, the ethylene is in a mixture with hydrogen.

Preferably, the pyrrole derivative corresponds to the general formula (I)

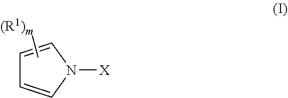

wherein:
R¹ is chosen independently from a halogen, a C₁-C₁₅ alkyl group, a C(O)R′ group, a COOR″ group, CCl₃, CF₃, R′ being chosen from H, a C₁-C₆ alkyl, a chlorine, a bromine, a fluorine; R″ being chosen from H, a C₁-C₆ alkyl;
m is an integer between 0 and 4;
X is chosen from a hydrogen, a lithium, a sodium, a potassium, a cesium, or an aluminum atom.

Preferably, the molar ratio between the total number of halogen atoms provided by the components of the catalytic composition and the sum of the aluminum atoms, denoted Altot, provided by the aluminum-based compound and the halogenated aluminum compound, denoted halo/Altot, is between 0.10 and 3.0, preferably between 0.15 and 2.5, preferably between 0.20 and 2.0.

Preferably, the aromatic additive is chosen from an aromatic ether and/or an aromatic hydrocarbon.

Preferably, the aromatic ether corresponds to the general formula (II) below:

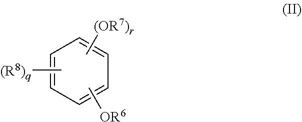

wherein:
R⁶ is chosen from a C₁-C₂₀ alkyl group, a C₃-C₂₀ cycloalkyl group, a C₂-C₂₀ alkenyl group, a C₅-C₂₀ aryl group optionally substituted with a C₁-C₆ alkyl group, or an aralkyl group;
R⁷ is chosen from a C₁-C₂₀ alkyl group, a C₃-C₂₀ cycloalkyl group, a C₂-C₂₀ alkenyl group, a C₅-C₂₀ aryl group optionally substituted with a C₁-C₆ alkyl group, or an aralkyl group;
R⁸ is chosen from a C₁-C₂₀ alkyl group, a C₃-C₂₀ cycloalkyl group, a C₂-C₂₀ alkenyl group, a C₅-C₂₀ aryl group optionally substituted with a C₁-C₆ alkyl group, or an aralkyl group;
q is an integer between 0 and 4,
r is an integer equal to 0 or 1.

Preferably, the molar ratio between the aromatic ether and the chromium-based metal precursor, denoted aromatic ether/Cr, is between 0.5 and 2000.0, preferably between 1.0 and 800.0, preferably between 2.0 and 600.0, preferably between 3.0 and 400.0.

Preferably, the aromatic hydrocarbon corresponds to the general formula (III) below

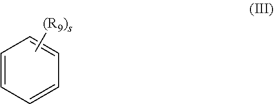

wherein:
R⁹ is chosen independently from a C₁-C₂₀ alkyl group, a C₃-C₂₀ cycloalkyl group,
s is an integer between 0 and 6.

Preferably, the molar ratio between the aromatic hydrocarbon and the chromium-based metal precursor, denoted aromatic hydrocarbon/Cr, is between 5.0 and 6000.0, preferably between 10.0 and 5500.0, preferably between 15.0 and 5000.0.

### Definitions and Abbreviations

It is specified that, throughout this description, the expressions “of between . . . and . . . ” and “comprising between . . . and . . . ” must be understood as including the limits mentioned.

The expression “Cx-Cy” for a hydrocarbon-based group means that said group comprises x to y carbon atoms.

A Cx-Cy alkyl group is understood to mean a hydrocarbon-based chain comprising between x and y carbon atoms, for example between 1 and 20 carbon atoms denoted C₁-C₂₀ alkyl, which is linear or branched, non-cyclic, cyclic or polycyclic, substituted or unsubstituted. For example, a C₁-C₈ alkyl is understood to mean an alkyl chosen from the methyl, ethyl, propyl, butyl, pentyl, cyclopentyl, hexyl, cyclohexyl, heptyl and octyl groups.

An alkoxy is understood to mean a monovalent group consisting of an alkyl group bonded to an oxygen atom such as the CH₃O—, C₂H₅O—, C₃H₇O— groups.

An aryloxy is understood to mean a monovalent group consisting of an aryl group bonded to an oxygen atom, such as the C₆H₅O— group.

An alkenyl group is understood to mean a hydrocarbon-based group comprising at least one double bond, said group being linear or branched and comprising from 2 to 20 carbon atoms, preferably from 2 to 6 carbon atoms, for example the ethenyl, vinyl, butenyl or prop-2-en-1-yl (allyl) group.

An aralkyl group is understood to mean a group comprising an alkyl group, a hydrogen atom of which is substituted with an aryl group, the alkyl and aryl groups being as defined above.

An aryl group is understood to mean a substituted or unsubstituted, fused or non-fused, monocyclic or polycyclic aromatic group comprising between 5 and 30 carbon atoms, denoted C₅-C₃₀ aryl.

Cr(III) is understood to mean a compound based on chromium with an oxidation state of +III. Similarly, Cr(II) and Cr(I) are understood to mean a compound based on chromium with an oxidation state respectively of +II and +I.

The molar ratio cited in the present invention, in particular relative to the chromium precursor, are understood and expressed relative to the number of moles of chromium contained in the catalytic composition.

### DETAILED DESCRIPTION OF THE INVENTION

Within the meaning of the present invention, the different embodiments presented can be used alone or in combination with one another, without any limit to the combinations.

For the purposes of the present invention, the various ranges of parameters for a given step, such as the pressure ranges and the temperature ranges, may be used alone or in combination. For example, for the purposes of the present invention, a preferred range of pressure values can be combined with a more preferred range of temperature values.

The present invention relates to an ethylene oligomerization process comprising, at a temperature between 90° C. and 190° C., simultaneously bringing ethylene into contact with the following components:
a metal precursor of chromium
a pyrrole derivative
an aluminum-based compound of general formula AlR²R³R⁴ wherein the R², R³ and R⁴ groups, which may be identical or different, are chosen independently from a hydrogen and C₁-C₂₀ alkyl, C₁-C₂₀ alkoxy and C₅-C₃₀ aryloxy groups,
at least one halogenated aluminum compound corresponding to the general formula AlₙR⁵ₒYₚ, wherein R⁵ is a C₁-C₂₀ alkyl group, Y is a halogen, n is an integer from 1 to 2, o is an integer from 1 to 3 and p is an integer from 1 to 3;
an aromatic additive;
optionally a solvent,
   said components forming in situ a catalytic composition.

One advantage of the process according to the invention is to simplify the implementation of the oligomerization process by a so-called in situ implementation in which the various components of the composition are brought into contact at the oligomerization reaction temperature and in the presence of ethylene, without any particular precaution.

Thus, the process according to the invention makes it possible to simplify the process scheme and therefore to limit the costs associated with the implementation of the oligomerization process while retaining high activity and high selectivity.

Another advantage of the process according to the invention is to limit the formation of polymer corresponding to the compounds (C12+) having a carbon number greater than or equal to 12, such as polyethylene.

Another advantage of the process according to the invention is to make it possible to dispense with the constraints linked to the preparation and possible storage of the catalytic composition.

The components of the catalytic composition are preferably brought into contact with ethylene, alone or in combination, advantageously according to the embodiments below.

In one preferred embodiment, the aluminum-based compound and the halogenated aluminum compound are mixed prior to them being brought into contact with ethylene, preferably at the time they are introduced into a reactor in which the oligomerization process according to the invention is carried out.

In one preferred embodiment, the pyrrole derivative is mixed with the aluminum-based compound and/or the halogenated aluminum compound prior to them being brought into contact with ethylene, preferably at the time they are introduced into the reactor.

In one preferred embodiment, the aluminum-based compound and the halogenated aluminum compound are mixed before being mixed with the pyrrole derivative, preferably in the presence of a solvent, prior to them being brought into contact with ethylene, preferably at the time they are introduced into the reactor.

In one preferred embodiment, the aluminum-based compound and the halogenated aluminum compound, the pyrrole derivative and the aromatic additive, preferably in the presence of a solvent, are mixed prior to them being brought into contact with ethylene, preferably at the time they are introduced into the reactor.

In one preferred embodiment, the metal precursor of chromium is mixed with the aromatic additive prior to them being brought into contact with ethylene, preferably at the time they are introduced into the reactor.

Preferably, the aluminum-based compound and the halogenated aluminum compound are introduced separately from the metal precursor of chromium for the simultaneous contacting thereof with ethylene. In other words, the aluminum-based compound and/or the halogenated aluminum compound are not mixed with the metal precursor of chromium prior to them being brought into contact with ethylene.

Advantageously, not all the components of the catalytic composition are mixed before being brought into contact with ethylene, that is to say that all the components are mixed at the time they are brought into contact with ethylene.

Preferably, the process is carried out at a pressure between from 0.5 to 15 MPa, preferably from 1.0 to 10.0 MPa, preferably between 1.5 and 8.0 MPa and very preferably between 2.0 and 7.0 MPa.

Preferably, the temperature for bringing the components of the catalytic composition into contact with ethylene is between 95° C. and 185° C., preferably between 100° C. and 160° C., preferably between 105° C. and 155° C., preferably between 110° C. and 150° C., preferably between 125° C. and 145° C.

Advantageously, the oligomerization process is carried out at a temperature between 95° C. and 185° C., preferably between 100° C. and 160° C., preferably between 105° C. and 155° C., preferably between 110° C. and 150° C., preferably between 125° C. and 145° C.

In one preferred embodiment, the temperature for bringing the catalytic composition into contact in the reactor is identical to the temperature of the oligomerization process.

Advantageously, the concentration of Cr used in the oligomerization process relative to the total volume of liquid used in the oligomerization reactor is between 10⁻¹² and 1 mol/L, and preferably between 10⁻⁹ and 0.4 mol/L.

Advantageously, the ethylene used in the oligomerization process is in a mixture with hydrogen. Preferably, the hydrogen is introduced in a ratio of between 0.1 and 5.0 wt % relative to the ethylene introduced, preferably between 0.2 and 4.0 wt %, preferably between 0.4 and 3.0 wt %, and very preferably between 0.5 and 2.0 wt %. Advantageously, the use of hydrogen as a mixture with ethylene makes it possible to increase the productivity and limit the molecular weight of the (C12+) polymers.

Advantageously, the oligomerization process is an ethylene trimerization process, preferably for the production of hex-1-ene.

Advantageously, the oligomerization reaction is carried out continuously.

The heat generated by the reaction may be removed via any means known to those skilled in the art.

The catalytic composition may be neutralized downstream of the reactor via any means known to those skilled in the art.

Preferably, the catalytic composition, the components of which are brought into contact with ethylene in the oligomerization process, comprises, and preferably consists of:
a chromium-based metal precursor,
a pyrrole derivative corresponding to the general formula (I)

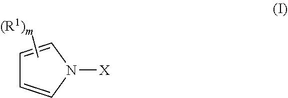

wherein:
R¹ is chosen independently from a halogen, a C₁-C₁₅ alkyl group, a C(O)R′ group, a COOR″ group, CCl₃, CF₃, R′ being chosen from H, a C₁-C₆ alkyl, a chlorine, a bromine, a fluorine; R″ being chosen from H, a C₁-C₆ alkyl;
m is an integer between 0 and 4;
X is chosen from a hydrogen, a lithium, a sodium, a potassium, a cesium, or an aluminum atom,
an aluminum-based compound of general formula AlR²R³R⁴ wherein the R², R³ and R⁴ groups, which may be identical or different, are chosen independently from a hydrogen and C₁-C₂₀ alkyl, C₁-C₂₀ alkoxy and C₅-C₃₀ aryloxy groups,
a halogenated aluminum compound corresponding to the general formula AlₙR⁵ₒYₚ wherein R⁵ is a C₁-C₂₀ alkyl group, Y is a halogen, n is an integer from 1 to 2, o is an integer from 1 to 3 and p is an integer from 1 to 3;
an aromatic additive;
optionally a solvent.

Chromium-Based Metal Precursor

One of the components of the catalytic composition brought into contact with ethylene in the process according to the invention is a chromium-based metal precursor, preferably chosen from a chromium(II) or chromium(III) salt.

Preferably, the chromium-based metal precursor may comprise one or more identical or different anions, preferably chosen from the group formed by halides, carboxylates, acetylacetonates, and alkoxy and aryloxy anions. The chromium compound may be a chromium(II) or chromium(III) salt, but also a salt with a different oxidation state that may comprise one or more identical or different anions, such as, for example, halides, carboxylates, acetylacetonates or alkoxy or aryloxy anions.

Preferably, the halide anions are chosen from chloride, bromide, fluoride or iodide.

Preferably, the carboxylate anions are chosen from the carboxylates having a C₃-C₂₀, preferably C₃-C₁₅, preferably C₄-C₁₂, preferably C₅-C₁₀ linear or branched alkyl chain, preferably said alkyl chain is unsubstituted or substituted by one or more fluorine, chlorine or bromine atoms.

Preferably, the alkoxy anions are chosen from the alkoxys having a C₁-C₂₀, preferably C₂-C₁₅, preferably C₃-C₁₂, preferably C₄-C₁₀ linear, branched, cyclic or non-cyclic alkyl chain, preferably said alkyl chain is unsubstituted or substituted by one or more fluorine, chlorine or bromine atoms.

Preferably, the aryloxy anions are chosen from the aryloxys having a C₅-C₃₀, preferably C₅-C₂₀, preferably C₆-C₁₅, preferably C₆-C₁₂ aryl group, preferably said aryl group is unsubstituted or substituted by one or more fluorine, chlorine or bromine atoms.

Preferably, the chromium-based metal precursor may be a chromium(III) compound, but a chromium(I) or chromium(II) compound may also be suitable. Mention may be made, as nonlimiting examples, of Cr(III) acetylacetonate, Cr(III) trifluoroacetylacetonate, Cr(III) hexafluoroacetylacetonate, Cr(III) acetate, Cr(III) 2-ethylhexanoate, Cr(III) heptanoate, Cr(III) naphthenate, Cr(III) chloride and Cr(III) bromide, taken alone or as a mixture, pure or diluted. The preferred metal precursors based on chromium are Cr(III) acetylacetonate, Cr(III) 2-ethylhexanoate and Cr(III) heptanoate.

Pyrrole Derivative

One of the components of the catalytic composition brought into contact with ethylene in the process according to the invention is a pyrrole derivative, preferably corresponding to the general formula (I)

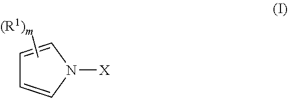

wherein:
R¹ is chosen independently from a halogen, a C₁-C₁₅ alkyl group, a C(O)R′ group, a COOR″ group, CCl₃, CF₃, R′ being chosen from H, a C₁-C₆ alkyl, a chlorine, a bromine, a fluorine; R″ being chosen from H, a C₁-C₆ alkyl;
m is an integer between 0 and 4;
X is chosen from hydrogen, lithium, sodium, potassium, cesium, or an aluminum atom.

Advantageously, R¹ is chosen independently from a fluorine, a chlorine, a C₁-C₁₀ alkyl group, a C(O)R′ group, a COOR″ group, CCl₃, CF₃, R′ being chosen from H, a methyl, an ethyl, a propyl, a butyl, a pentyl, a cyclohexyl, a chlorine, a fluorine. Preferably, R¹ is chosen independently from a fluorine, a chlorine, a C₁-C₆ alkyl group, a C(O)R′ group, a COOR″ group, CCl₃, CF₃, R′ being chosen from H, a methyl, an ethyl, a propyl, a butyl, R″ being chosen from H, a methyl, an ethyl, a propyl, a butyl.

Preferably, m is an integer equal to 0, 1, 2, 3 or 4, preferably m is equal to 0 or 2.

Preferably, the pyrrole derivative is chosen from tetrahydroindole, 2,5-dimethylpyrrole, 3,4-dimethylpyrrole, 3,4-dichloropyrrole, 2,3,4,5-tetrachloropyrrole, 2,4-dimethyl-3-ethylpyrrole, pyrrole-2-carboxylic acid, 2-acetylpyrrole, pyrrole-2-carboxaldehyde, 3-acetyl-2,4-dimethylpyrrole, ethyl 2,4-dimethyl-5-(ethoxycarbonyl)-3-pyrroleproprionate, ethyl 3,5-dimethyl-2-pyrrolecarboxylate, alone or as mixtures. Preferably, the pyrrole derivative is chosen from pyrrole (C₄H₅N) and 2,5-dimethylpyrrole.

Preferably, the molar ratio of the compound derived from pyrrole to the chromium-based metal precursor, denoted DMP/Cr, is between 0.5 and 40.0, preferably between 1.0 and 10.0, preferably between 1.5 and 8.0, preferably between 2.0 and 5.0 and very preferably between 2.5 and 4.5.

Aluminum-Based Compound

One of the components of the catalytic composition brought into contact with ethylene in the process according to the invention is an aluminum-based compound of general formula AlR²R³R⁴ wherein the R², R³ and R⁴ groups, which may be identical or different, are chosen independently from a hydrogen and C₁-C₂₀ alkyl, C₁-C₂₀ alkoxy and C₅-C₃₀ aryloxy groups.

When at least one of the R², R³ and R⁴ groups is chosen from alkyl and alkyloxy groups, said alkyl and alkyloxy groups preferably comprise between 1 and 15 carbon atoms, preferably between 1 and 10 carbon atoms, preferably between 1 and 6 carbon atoms and very preferably between 1 and 4 carbon atoms.

When at least one of the R², R³ and R⁴ groups is chosen from aryloxy groups, said aryloxy groups preferably comprise between 5 and 20 carbon atoms, preferably between 5 and 15 carbon atoms and preferably between 5 and 10 carbon atoms.

Preferably, one of the R², R³ and R⁴ groups is a hydrogen.

Preferably, the R², R³ and R⁴ groups are identical.

Preferably, at least one of the R², R³ and R⁴ groups is chosen independently from the others from alkyl and alkyloxy groups. Preferably said alkyl and alkyloxy groups are chosen from methyl, ethyl, propyl, n-butyl, iso-butyl, sec-butyl, tert-butyl, pentyl, hexyl, heptyl, octyl, and from the corresponding alkyloxy groups.

Preferably, at least one of the R², R³ and R⁴ groups is chosen independently from the others from aryloxy groups. Very preferably, said aryloxy group is phenoxy (C₆H₅O—).

Advantageously, the aluminum-based compound(s) of general formula AlR²R³R⁴ are chosen from trimethylaluminum, triethylaluminum, tri-n-propylaluminum, triisopropylaluminum, tri-n-butylaluminum, triisobutylaluminum, tri-tert-butylaluminum, trihexylaluminum, trioctylaluminum, diethylethoxyaluminum and dimethylethoxyaluminum. Preferably, the aluminum-based compound is triethylaluminum or triisobutylaluminum.

Preferably, the molar ratio of the aluminum-based compound of general formula AlR²R³R⁴ to the chromium-based metal precursor, denoted AI/Cr, is between 1.0 and 100.0, preferably between 2.0 and 90.0, preferably between 3.0 and 80.0, preferably between 4.0 and 60.0, preferably between 5.0 and 50.0, preferably between 6.0 and 40.0, preferably between 7.0 and 30.0, very preferably between 8.0 and 20.0, and more preferably still between 9.0 and 15.0.

Halogenated Aluminum Compound

One of the components of the catalytic composition brought into contact with ethylene in the process according to the invention is a halogenated aluminum compound corresponding to the general formula AlₙR⁵ₒYₚ wherein
R⁵ is a C₁-C₂₀ alkyl group,
Y is a halogen,
n is an integer from 1 to 2,
o is an integer from 1 to 3, and
p is an integer from 1 to 3.

Preferably, R⁵ is a C₁-C₁₅, preferably C₁-C₁₀, preferably C₁-C₆ alkyl. Preferably, R⁵ is chosen from methyl, ethyl, propyl, n-butyl, iso-butyl, sec-butyl, tert-butyl, pentyl, hexyl, heptyl, octyl.

Preferably, Y is chosen from a fluorine, a chlorine or a bromine. Preferably, Y is a chlorine or a bromine.

Advantageously, the halogenated aluminum compound is chosen from the group formed by methylaluminum dichloride (MeAlCl₂), ethylaluminum dichloride (EtAlCl₂), ethylaluminum sesquichloride (Et₃Al₂C₁₃), diethylaluminum chloride (Et₂AlCl), diisobutylaluminum chloride (iBu₂AlCl) and isobutylaluminum dichloride (iBuAlCl₂), taken alone or as a mixture. Very preferably, the halogenated aluminum compound is ethylaluminum dichloride (EtAlCl₂) and diethylaluminum chloride (Et₂AlCl).

Preferably, the molar ratio of the halogenated aluminum compound of general formula AlₙR⁵ₒYₚ to the chromium-based metal precursor, denoted AlY/Cr, is between 1.0 and 100.0, preferably between 1.5 and 80.0, preferably between 2.0 and 60.0, preferably between 2.5 and 50.0, preferably between 3.0 and 40.0, preferably between 3.5 and 30.0, preferably between 4.0 and 20.0, very preferably between 4.5 and 15.0, and more preferably still between 5.0 and 12.0.

In one preferred embodiment, the molar ratio between the total number of halogen atoms provided by the components of the catalytic composition, preferably chlorine or bromine, and the sum of the aluminum atoms (denoted Altotal or Altot) provided by the aluminum-based compound (Al) and the halogenated aluminum compound (AlY), denoted halo/Altot, and in particular Cl/Altot when the halogen is chlorine and Br/Altot when the halogen is bromine, is between 0.10 and 3.0, preferably between 0.15 and 2.5, preferably between 0.20 and 2.0, preferably between 0.25 and 1.5 and very preferably between 0.30 and 1.0.

Advantageously, when the catalytic composition has Halo/Al molar ratios within the ranges defined above, said composition has a better activity and hex-1-ene selectivity for the ethylene trimerization.

Aromatic Additive

One of the components of the catalytic composition brought into contact with ethylene in the process according to the invention is at least one aromatic additive. Advantageously, the aromatic additive is an aromatic ether and/or an aromatic hydrocarbon. Preferably, the aromatic additive is chosen from the compounds corresponding to the general formulae (II) and/or (III) below.

The aromatic additive may be an aromatic ether corresponding to the general formula (II) below:

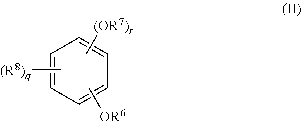

wherein:
R⁶ is chosen from a C₁-C₂₀ alkyl group, a C₃-C₂₀ cycloalkyl group, a C₂-C₂₀ alkenyl group, a C₅-C₂₀ aryl group optionally substituted with a C₁-C₆ alkyl group, or an aralkyl group;
R⁷ is chosen from a C₁-C₂₀ alkyl group, a C₃-C₂₀ cycloalkyl group, a C₂-C₂₀ alkenyl group, a C₅-C₂₀ aryl group optionally substituted with a C₁-C₆ alkyl group, or an aralkyl group;
R⁸ is chosen from a C₁-C₂₀ alkyl group, a C₃-C₂₀ cycloalkyl group, a C₂-C₂₀ alkenyl group, a C₅-C₂₀ aryl group optionally substituted with a C₁-C₆ alkyl group, or an aralkyl group;
q is an integer between 0 and 4,
r is an integer equal to 0 or 1.

Preferably, R⁶ is chosen from a C₁-C₁₀ alkyl group, a C₃-C₁₀ cycloalkyl group, a C₂-C₁₀ alkenyl group, a C₅-C₁₅ aryl group. Preferably, R⁶ is chosen from a C₁-C₆ alkyl group, a C₃-C₆ cycloalkyl group, a C₂-C₈ alkenyl group, a C₅-C₁₂ aryl group. Preferably, R⁶ is chosen from methyl, ethyl, n-propyl, isopropyl, n-butyl, tert-butyl, pentyl, cyclohexyl, benzyl, phenyl, 2-methylphenyl, 2,6-dimethylphenyl or 2,4,6-trimethylphenyl groups. Very preferably, R⁶ is chosen from methyl, ethyl, n-propyl, isopropyl, n-butyl, tert-butyl, cyclohexyl, benzyl, phenyl, 2-methylphenyl, 2,6-dimethylphenyl or 2,4,6-trimethylphenyl groups.

Preferably, R⁷ is chosen from a C₁-C₁₀ alkyl group, a C₃-C₁₀ cycloalkyl group, a C₂-C₁₀ alkenyl group, a C₅-C₁₅ aryl group. Preferably, R⁷ is chosen from a C₁-C₆ alkyl group, a C₃-C₆ cycloalkyl group, a C₂-C₈ alkenyl group, a C₅-C₁₂ aryl group. Preferably, R⁷ is chosen from a methyl, ethyl, n-propyl, isopropyl, n-butyl, tert-butyl, pentyl, cyclohexyl, benzyl, phenyl, 2-methylphenyl, 2,6-dimethylphenyl or 2,4,6-trimethylphenyl group. Very preferably, R⁷ is chosen from a methyl, ethyl, n-propyl, isopropyl, n-butyl, tert-butyl, cyclohexyl, benzyl, phenyl, 2-methylphenyl, 2,6-dimethylphenyl or 2,4,6-trimethylphenyl group.

Preferably, R⁸ is chosen from a C₁-C₁₀ alkyl group, a C₃-C₁₀ cycloalkyl group, a C₂-C₁₀ alkenyl group, a C₅-C₁₅ aryl group. Preferably, R⁸ is chosen from a C₁-C₆ alkyl group, a C₃-C₆ cycloalkyl group, a C₂-C₈ alkenyl group, a C₅-C₁₂ aryl group. Preferably, R⁸ is chosen from a methyl, ethyl, n-propyl, isopropyl, n-butyl, tert-butyl, pentyl, cyclohexyl, benzyl, phenyl, 2-methylphenyl, 2,6-dimethylphenyl or 2,4,6-trimethylphenyl group. Very preferably, R⁸ is chosen from a methyl, ethyl, n-propyl, isopropyl, n-butyl, tert-butyl, cyclohexyl, benzyl, phenyl, 2-methylphenyl, 2,6-dimethylphenyl or 2,4,6-trimethylphenyl group.

Preferably, q is equal to 0, 1 or 2.

Advantageously, r is equal to 0 or 1. Preferably, r is equal to 0.

When r is equal to 1, the OR⁷ group is in the ortho, meta or para position relative to the OR⁶ group. Preferably, the OR⁷ group is in the ortho position.

When q is equal to 1 or 2, the OR⁸ group or groups, which may be identical or different, are in the ortho, meta or para position relative to the OR⁶ group. Preferably, the R⁸ group(s) are in the ortho position.

Preferably, the aromatic additive of ether type is chosen from methoxybenzene (or anisole), 2-methylanisole, 3-methylanisole, 4-methylanisole, 2-chloroanisole, 3-chloroanisole, 4-chloroanisole, 3,5-dichloroanisole, 2,6-dichloroanisole, 1,2-dimethoxybenzene, 1,3-dimethoxybenzene, 1,4-dimethoxybenzene, 2,3-dimethylanisole, ethoxybenzene, diphenyl ether, 1-methoxynaphthalene, 2-methoxynaphthalene, 2,7-dimethoxynaphthalene, 1,3-dimethoxynaphthalene. Preferably, the aromatic additive of ether type is methoxybenzene, 1,2-dimethoxybenzene or 2,3-dimethylanisole.

Preferably, the molar ratio between the aromatic ether and the chromium-based metal precursor, denoted aromatic ether/Cr, is between 0.5 and 2000.0, preferably between 1.0 and 800.0, preferably between 2.0 and 600.0, preferably between 3.0 and 400.0, preferably between 5.0 and 300.0, preferably between 7.0 and 200.0, preferably between 10.0 and 150.0, preferably between 12.0 and 100.0, preferably between 15.0 and 90.0, more preferably between 20.0 and 80.0, and more preferably still between 30.0 and 70.0.

The aromatic additive may be an aromatic hydrocarbon corresponding to the general formula (III) below:

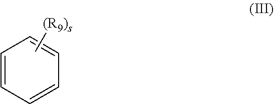

wherein:
R⁹ is chosen independently from a C₁-C₂₀ alkyl group, a C₃-C₂₀ cycloalkyl group,
s is an integer between 0 and 6.

Preferably, R⁹ is chosen from a C₁-C₁₀ alkyl group, preferably a C₁-C₆ alkyl group, a C₃-C₁₀ cycloalkyl group, preferably a C₃-C₆ cycloalkyl group. Preferably, R⁹ is chosen from methyl, ethyl, n-propyl, isopropyl, n-butyl, tert-butyl, pentyl, cyclohexyl groups.

Preferably, s is equal to 0, 1, 2, 3, 4, 5 or 6. Preferably, s is between 1 and 4 and more preferably s is equal to 1, 2 or 3.

Preferably, the aromatic hydrocarbon additive is chosen from benzene, toluene, ethylbenzene, diethylbenzene, ortho-xylene, meta-xylene, para-xylene, styrene, cumene, alone or as a mixture. Preferably, the aromatic hydrocarbon additive is chosen from toluene, ethylbenzene and o-xylene.

In one particular embodiment, the catalytic composition used in a process according to the invention may comprise at least one aromatic ether of formula (II) and at least one aromatic hydrocarbon of formula (III) as additives.

Preferably, the molar ratio between the aromatic hydrocarbon additive and the chromium-based metal precursor, denoted aromatic hydrocarbon/Cr, is between 5.0 and 6000.0, preferably between 10.0 and 5500.0, preferably between 15.0 and 5000.0, preferably between 20.0 and 4500.0, preferably between 25.0 and 4000.0, preferably between 30.0 and 3500.0, preferably between 35.0 and 3000.0, preferably between 40.0 and 2500.0, preferably between 45.0 and 2000.0, preferably between 50.0 and 1700.0, preferably between 55 and 1500.0, preferably between 60.0 and 1200.0, preferably between 70.0 and 1000.0, preferably between 80.0 and 900.0, preferably between 90.0 and 800, preferably between 100 and 700, preferably between 150 and 600, and very preferably between 200 and 500.

Optional Solvent

One of the components of the catalytic composition brought into contact with ethylene in the process according to the invention may additionally be at least one solvent. Preferably, said solvent is chosen from organic solvents and in particular from saturated, unsaturated, cyclic or non-cyclic hydrocarbons.

The solvent(s) is (are) advantageously chosen from halogenated solvents and saturated or unsaturated, cyclic or non-cyclic hydrocarbons comprising between 1 and 20 carbon atoms, preferably between 1 and 15 carbon atoms and preferably between 4 and 15 carbon atoms. Preferably, the solvent is chosen from pentane, hexane, cyclohexane, methylcyclohexane, heptane, butane or isobutane, cycloocta-1,5-diene, dichloromethane, dichloroethane, chlorobenzene, dichlorobenzene, methanol, ethanol, pure or as a mixture. Preferably, the solvent is chosen from hexane, cyclohexane and methylcyclohexane.

In the case where the solvent is an unsaturated hydrocarbon, it may be advantageously chosen from the products of the oligomerization reaction.

The following examples illustrate the invention without limiting the scope thereof.

### EXAMPLES

The content of solvent is the ratio by weight of the total flow rate of injected solvent to the total flow rate of injected ethylene in the process.

The distribution of olefins obtained by the process is given as percentage of butenes (% C4), of hexenes (% C6), of octenes (% C8), of decenes (% C10) and of olefins having a carbon number equal to or greater than 12.

The %1-C6 corresponds to the selectivity for hex-1-ene in the hexenes cut.

### Examples 1 to 5: Use of the Catalytic System in Continuous Mode

Formulation of the Catalytic System:

Solution 1: In a prepared measuring cylinder, under stirring, triethylaluminum (final concentration=0.00825 mol/L) is introduced, followed by the diethylaluminum chloride (final concentration=0.006 mol/L) and the 2,5-dimethylpyrrole ligand (final concentration=0.00225 mol/L). The total volume of the solution is 1.5 L.

Solution 2: In a prepared measuring cylinder, under stirring, the chromium precursor Cr(2-EH)₃ (xx g, final concentration=0.0075 mol/L) and the anisole (50 eq, 0.03750 mol/L) are diluted in cyclohexane. The total volume of the solution is 1.5 L.

Use of the Catalytic System

The solutions of the ligand (2,5-dimethylpyrrole) combined with the co-catalysts (triethylaluminum and diethylaluminum chloride) (Solution 1) and of the chromium precursor (Cr(2-EH)₃) combined with the aromatic compound (anisole and/or o-xylene) (Solution 2), diluted in cyclohexane (water measured below 10 ppm), are introduced into the reactor under flow rate control by means of a system of pumps. The two lines for introducing the solutions are independent of one another. The points for injecting these two solutions into the reactor are located directly in the liquid phase.

The ethylene (optionally mixed with 0.5 wt % hydrogen) is introduced into the reactor under pressure control. The reaction takes place in a 250 ml gas/liquid CSTR reactor. It is equipped with a 5-point thermometer tube enabling the regulation of the liquid level at various levels, with oil-bath heating (controlled at the temperature of the liquid phase) and with mechanical stirring (from 0 to 1500 rpm).

The liquid phase containing the dissolved ethylene, optionally the dissolved hydrogen, and the products formed is discharged from the reactor via a dip tube and a discharge valve, said phase is neutralized and separated.

The gas phase is quantified and qualified by gas chromatography (GC), the liquid phase is weighed, neutralized and qualified by GC. The results obtained are presented in the table below, the amounts of reactants are indicated in equivalents (denoted eq) relative to the amount of chromium used.

| Examples | 1 | 2 | 3 | 4 | 5 |
|---|---|---|---|---|---|
| Pressure (MPa) | 6.0 | 6.0 | 4.0 | 4.0 | 6.0 |
| Temperature (° C.) | 135 | 135 | 115 | 135 | 135 |
| FEEDSTOCK | C₂H₄ | C₂H₄/H₂ | C₂H₄/H₂ | C₂H₄ | C₂H₄ |
| Content of solvent | 62 | 62 | 66 | 75 | 64 |
| Residence time (h) | 0.50 | 0.50 | 0.65 | 0.55 | 0.44 |
| 2,5-DMP | 3 | 3 | 3 | 3 | 3 |
| Et₃Al | 11 | 10 | 10 | 10 | 11 |
| (eq) | | | | | |
| Et₂AlCl | 8 | 9 | 8 | 9 | 8 |
| [symbol] | | | | | |
| Additive (eq) | Anisole | Anisole | Anisole | o-xylene | o-xylene |
| | (50) | (50) | (50) | (500) | (500) |
| Conc. Cr reactor | 1.1 | 1.2 | 1.0 | 1.2 | 1.0 |
| (ppm) | | | | | |
| Reaction time | 16.1 | 15.6 | 6.3 | 14.8 | 17.8 |
| Productivity | 207510 | 202548 | 249249 | 136.587 | 222300 |
| % C4 (%) | 0.0 | 0.0 | 0.0 | 0.0 | 0.0 |
| % C6 (%) | 94.8 | 94.6 | 93.8 | 95.9 | 95.3 |
| %1-C6 (%) | 99.4 | 99.3 | 99.5 | 99.5 | 99.6 |
| % C8 (%) | 0.2 | 0.2 | 0.2 | 0.2 | 0.2 |
| % C10 (%) | 4.0 | 4.2 | 5.2 | 3.1 | 3.4 |
| % C12+ (%) | 1.0 | 1.0 | 0.7 | 0.9 | 1.0 |

[symbol]

indicates data missing or illegible when filed

Examples 1 to 5 clearly show that an “in situ” process according to the present invention using a catalytic composition comprising an aromatic additive makes it possible to obtain very good productivities and excellent selectivities in favor of hex-1-ene.

### Examples 6 to 8: Use of the Catalytic System in Semi-Open Batch Mode

Examples 6 to 8 were carried out in a stainless steel autoclave with a working volume of 250 ml equipped with a jacket enabling the temperature to be regulated by circulation of oil.

The reactor is predried under vacuum and placed under an ethylene atmosphere.

Solution 1: In a Schlenk flask placed under an inert atmosphere and under stirring, while observing the desired ratios, a solution of triethylaluminum (0.0365 mol/l) in cyclohexane is introduced, followed by a solution of diethylaluminum chloride (0.0243 mol/l) in cyclohexane and by a solution of 2,5-dimethylpyrrole (0.0301 mol/l) in cyclohexane. The mixture thus obtained is stirred under argon for 15 minutes.

Solution 2: The chromium precursor Cr(2-EH)₃ (20 μmol) and the anisole (50 eq) are diluted in cyclohexane. The total volume of the solution is 18 ml.

The reactor is predried under vacuum and placed under an ethylene atmosphere. The cyclohexane is introduced into the reactor under an ethylene atmosphere and then solution 1 containing Et₃Al (9-13 eq/Cr), Et₂AlCl (6-10 eq/Cr) and 2,5-DMP (3 to 4 eq/Cr) is added. Solution 2 is introduced into the injection airlock. The total volume of liquid is 75 ml. Once the temperature of the reactor has been brought to the test temperature, the solution contained in the injection airlock is injected into the reactor under ethylene pressure. The pressure is adjusted to the test pressure. The ethylene consumption is monitored until 35 g of ethylene has been introduced. The supply of ethylene is then cut and the reactor is cooled and degassed. The gas phase is quantified and qualified by gas chromatography (GC), the liquid phase is weighed, neutralized and qualified by GC.

| | Et₃Al | Et₂AlCl | | Time | Productivity | | % C6 | | | HBP |
|---|---|---|---|---|---|---|---|---|---|---|
| Example | (eq) | (eq) | Cl/Al | (min) | (g/(gCr · h)) | C4 | (% 1C6) | % C8 | % C10 | % C12+ |
| 6 | 13 | 6 | 0.32 | 26 | 84400 | 0.6 | 88.3 | 0.9 | 5.6 | 4.6 |
| | | | | | | | (98.8) | | | |
| 7 | 11 | 8 | 0.42 | 38 | 50731 | 0.3 | 92.2 | 0.5 | 4.6 | 2.4 |
| | | | | | | | (99.2) | | | |
| 8 | 9 | 10 | 0.53 | 55 | 21423 | 0.3 | 94.7 | 0.3 | 3.1 | 1.6 |
| | | | | | | | (99.4) | | | |

Operating conditions: Cr(2-EH)₃ (20 μmol); 2,5-DMP (3 eq); anisole (50 eq); cyclohexane (75 ml); 135° C., 50 bar

It clearly appears, in view of examples 6 to 8, that the process according to the invention using a catalytic composition having a precise ratio between the total number of chlorine atoms provided by the components of the catalytic composition and the sum of the aluminum atoms (Cl/Al) in an oligomerization process advantageously makes it possible to adjust the performance of the composition, in particular in terms of selectivity and productivity.

1. An ethylene oligomerization process comprising, at a temperature between 90° C. and 190° C., simultaneously bringing ethylene into contact with the following components:
a metal precursor of chromium
a pyrrole derivative
an aluminum-based compound of general formula AlR²R³R⁴ wherein the R², R³ and R⁴ groups, which may be identical or different, are chosen independently from a hydrogen and C₁-C₂₀ alkyl, C₁-C₂₀ alkoxy and C₅-C₃₀ aryloxy groups,
at least one halogenated aluminum compound corresponding to the general formula AlₙR⁵ₒYₚ, wherein R⁵ is a C₁-C₂₀ alkyl group, Y is a halogen, n is an integer from 1 to 2, o is an integer from 1 to 3 and p is an integer from 1 to 3;
an aromatic additive,
said components forming in situ a catalytic composition.

2. The process as claimed in claim 1, wherein the aluminum-based compound and the halogenated aluminum compound are mixed prior to them being brought into contact with ethylene.

3. The process as claimed in claim 1, wherein the aluminum-based compound and the halogenated aluminum compound are mixed before being mixed with the pyrrole derivative prior to them being brought into contact with ethylene.

4. The process as claimed in claim 1, wherein the aluminum-based compound and the halogenated aluminum compound, the pyrrole derivative and the aromatic additive are mixed prior to them being brought into contact with ethylene.

5. The process as claimed in claim 1, wherein the metal precursor of chromium and the aromatic additive are mixed prior to them being brought into contact with ethylene.

6. The process as claimed in claim 1, wherein all the components are mixed at the time they are brought into contact with ethylene.

7. The process as claimed in claim 1, wherein the temperature for bringing the components into contact with ethylene is between 95° C. and 185° C., preferably between 100° C. and 160° C.

8. The process as claimed in claim 1, wherein the ethylene is in a mixture with hydrogen.

9. The process as claimed in claim 1, wherein the pyrrole derivative corresponds to the general formula (I) 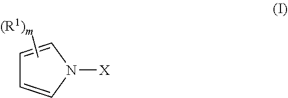
wherein:
R¹ is chosen independently from a C₁-C₁₅ alkyl group, a C(O)R′ group, a COOR″ group, CCl₃, CF₃, R′ being chosen from H, a C₁-C₆ alkyl, a chlorine, a bromine, a fluorine; R″ being chosen from H, a C₁-C₆ alkyl;
m is an integer between 0 and 4;
X is chosen from hydrogen, lithium, sodium, potassium, cesium, or an aluminum atom.

10. The process as claimed in claim 1, wherein the molar ratio between the total number of halogen atoms provided by the components of the catalytic composition and the sum of the aluminum atoms, denoted Altot, provided by the aluminum-based compound and the halogenated aluminum compound, denoted halo/Altot, is between 0.10 and 3.0, preferably between 0.15 and 2.5, preferably between 0.20 and 2.0.

11. The process as claimed in claim 1, wherein the aromatic additive is chosen from an aromatic ether and/or an aromatic hydrocarbon.

12. The process as claimed in claim 11, wherein the aromatic ether corresponds to the general formula (II) below: 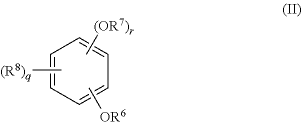
wherein:
R⁶ is chosen from a C₁-C₂₀ alkyl group, a C₃-C₂₀ cycloalkyl group, a C₂-C₂₀ alkenyl group, a C₅-C₂₀ aryl group optionally substituted with a C₁-C₆ alkyl group, or an aralkyl group;
R⁷ is chosen from hydrogen, a C₁-C₂₀ alkyl group, a C₃-C₂₀ cycloalkyl group, a C₂-C₂₀ alkenyl group, a C₅-C₂₀ aryl group optionally substituted with a C₁-C₆ alkyl group, or an aralkyl group;
R⁸ is chosen from a C₁-C₂₀ alkyl group, a C₃-C₂₀ cycloalkyl group, a C₂-C₂₀ alkenyl group, a C₅-C₂₀ aryl group optionally substituted with a C₁-C₆ alkyl group, or an aralkyl group;
q is an integer between 0 and 4,
r is an integer equal to 0 or 1.

13. The process as claimed in claim 11, wherein the molar ratio between the aromatic ether and the chromium-based metal precursor, denoted aromatic ether/Cr, is between 0.5 and 2000.0, preferably between 1.0 and 800.0, preferably between 2.0 and 600.0, preferably between 3.0 and 400.0.

14. The process as claimed in claim 11, wherein the aromatic hydrocarbon corresponds to the general formula (III) below 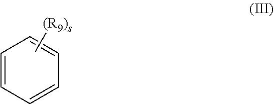
wherein:
R⁹ is chosen independently from a C₁-C₂₀ alkyl group, a C₃-C₂₀ cycloalkyl group,
s is an integer between 0 and 6.

15. The process as claimed in claim 11, wherein the molar ratio between the aromatic hydrocarbon and the chromium-based metal precursor, denoted aromatic hydrocarbon/Cr, is between 5.0 and 6000.0, preferably between 10.0 and 5500.0, preferably between 15.0 and 5000.0.
